(19) United States
(12) Patent Application Publication
(10) Pub. No.: US 2006/0135852 A1
(43) Pub. Date: Jun. 22, 2006

(54) Rotatable holder assembly for a surgical retractor blade

(76) Inventors: Tibor Koros, Moorpark, CA (US); Gabriel Koros, Thousand Oaks, CA (US)

Correspondence Address: FULWIDER PATTON, 6060 CENTER DRIVE, 10TH FLOOR, LOS ANGELES, CA, 90045, US

(21) Appl. No.: 11/086,864

(22) Filed: Mar. 22, 2005

Related U.S. Application Data
(63) Continuation-in-part of application No. 29/219,557, filed on Dec. 20, 2004.

(51) Int. Cl. A61B 1/32

(52) U.S. Cl. 600/234

(57) ABSTRACT

A hand held computer for law enforcement officers in which to assist their performance of their daily duties. The hand held computer includes a microprocessor, a keyboard and a data input system for entering data into the microprocessor, a display screen and a data output circuit for displaying data from the microprocessor, and a data storage circuit for storing data converted to the microprocessor, and a plurality of control keys for operating in a plurality of modes. The hand held computer further includes a data storage circuit for controlling at least three law conversion functions having at least three law entry keys. The first law entry key is a traffic statute key for inserting a statute number inquiry or a key word(s) inquiry related to the statute. The second law entry key is a municipal statute key for inserting a statute number inquiry or a key word(s) inquiry related to the statute. The third law entry key being a criminal statute key for inserting a statute inquiry or a key word(s) inquiry related to the statute. Each of the law entry keys are for displaying on the display screen a traffic, a municipal or a criminal statute response, respectively, to the inquiry.

### CROSS-REFERENCES TO RELATED APPLICATIONS

This is a continuation-in-part of U.S. Ser. No. 29/219,557 filed on Dec. 20, 2004.

### BACKGROUND OF THE INVENTION

The present invention relates generally to mechanisms for holding a surgical retractor blade and, more particularly, to a holder assembly for holding and positioning a surgical retractor blade which allows the retractor blade to be retracted and rotated to enable both lateral and angular adjustment of the retractor blade relative to a surgical site.

Various surgical retraction systems have been developed over the years for use in surgical operations which require access to internal organs and bone structures. Surgical retraction systems are used to hold back tissue and expose the area in which the surgical operation is to be performed. In many surgical retraction systems, some type of a retractor device is utilized to hold the retractor blade which is used to engage and hold the patient's tissue. The retractor device is usually designed to be mounted a support frame mounted to the operating room table above the patient. The support frame usually includes a number of rails erected above the surgical site which forms a ring or oval support structure that remains stationary throughout the surgical procedure. A number of retractor devices with retractor blades can be placed along the support frame at appropriate positions to allow the retractor blades to retract the patient's tissue and expose the area for the surgical procedure. Due to a number of factors such as the size and location of the surgical site, along with variation in patient size, the desired exposure is not always directed to the center of the operation site, which many times requires retractor blades to be positioned, elevated or pushed up or down on the margins of the surgical incision. Accordingly, it is very important that the retractor device and retractor blade be designed for ease in repositioning into different angular positions as may be needed by the surgical staff in order to properly expose the surgical site for surgery. Also, the retractor device should allow the retractor blade to be positioned without the need to remove the retractor device from the surgical support frame.

There are various types of retraction mechanisms which can be utilized in conjunction with a surgical support frame. Most rotator blades are supplied with a rod or stem portion which has the surgical blade attached to one end. The mechanism for holding the rod or stem of the retractor blade usually supplies a compressive force to clamp the rod or stem therein. Other systems may utilize pawl and ratchet mechanisms for holding the stem. In either system, the holding mechanism should be designed such that it is easy to manipulate the stem between clamped and unclamped positions and should be easily readjustable to achieve the desired tension necessary to hold back the tissue to expose the area to be surgically treated.

Various types of retractor devices which utilize clamping mechanisms have been developed for retraction systems and include those disclosed in U.S. Pat. No. 5,727,899 (Dobrovolny); U.S. Pat. No. 5,792,046 (Dobrovolny); U.S. Pat. No. 5,888,197 (Mulac); U.S. Pat. No. 6,017,008 (Farley); and U.S. Pat. No. 6,277,069 (Gray). These patents generally relate to the basic concept of holding two rod sections in an adjustable and fixable angular relationship relative to one another when placed in a locked position. One of the rod sections is usually a retractor handle that has a retractor blade mounted at one end and is laterally adjustable to engage the tissue at the surgical site. The other rod section is usually the rail of the support frame which allows the retractor blade to be movably placed around the area of the surgical site. Different types of clamps are disclosed in these patents which are designed to engage these rod sections. Many of these devices include a universal joint mechanism which permits quick tightening of the retractor clamp for ease of movement on the rail system along with ease of movement of the retractor blade.

Another type of retractor device which utilizes a pawl and ratchet mechanism is shown in U.S. Pat. No. 6,620,097 (Bookwalter). This patent describes a tilt ratchet mechanism which must utilize a number of different components that must be pivotally or rotationally connected to each other to create a composite tilt mechanism for holding the stem of the surgical retractor blade. This combination of separate components that pivot or rotate relative to each other can create a composite device that is bulky and has a large profile which could create an unwanted obstruction to the surgeon when reaching into the surgical site to perform the required surgery. Also, a combination of numerous moving components can possibly cause the composite device to become somewhat unbalanced on the surgical support frame as the surgeon positions the retractor blade. Accordingly, it is usually desired to have a simple yet effective holding mechanism which minimizes the number of moving components yet has sufficient gripping strength to hold onto the retractor stem as tensioning force is placed on the retractor blade to retract the patient's tissue. Additionally, more separate and moving parts associated with a holding mechanism generally increases the manufacturing cost for such a mechanism.

What has been needed is an improved apparatus which provides the force needed to maintain the surgical retractor blade in a clamped position while allowing the orientation of the retractor blade to be easily reposition by the surgical staff in order to properly expose the surgical site. In this regard, it would be beneficial if the holder apparatus not only allows the retractor blade to be moved in and out of the surgical site, as is needed, but also to be angularly adjusted, or rotated, to allow the retractor blade to be angularly adjusted while on the surgical support frame. Accordingly, such a holder apparatus should enhance the ability of the surgical staff to rotate the retractor blade and provide the surgeon with enhanced ability to control the amount of exposure in the surgical site. Such an apparatus should be compact, if possible, to minimize its size and prevent a large obstruction to be placed on the surgical support frame. It also would be beneficial if the holding apparatus has only a minimum number of moving components. The present invention satisfies these and other needs.

### SUMMARY OF THE INVENTION

The present invention provides a rotatable holder assembly which can be used to hold a surgical retractor blade on a surgical frame. The present invention can be used separately to hold the surgical blade, or in combination with other positioning devices to create a multi-positionable apparatus for accessing a surgical site. The present invention is sturdy, readily adjustable, easy to use and can be made from biocompatible material, such as stainless steel, which can be easily sterilized. The present invention is practical and particularly useful in the medical field as part of a surgical retraction apparatus used to provide exposure of an operative site.

In one aspect, the rotatable retractor blade holder of the present invention provides multidimensional movement and placement of a surgical retractor blade on a typical surgical retractor frame. Typically, a retractor blade includes an elongate stem portion and a surgical blade used to engage and hold the patient's tissue. In use, the surgeon simply moves the stem in a selected lateral movement to move the blade relative to a surgical site. This lateral movement of the stem in turn adjusts the amount of tension placed on the outer margin of the patient's tissue in order to create an anatomical surgical opening in which the surgeon performs the particular surgery. The rotatable retractor blade holder is also rotatable when mounted on the surgical frame to allow the surgeon to rotate both the stem and surgical blade about the longitudinal axis of the stem to position the blade as may be needed within the surgical site. This allows the rotation of the stem and retractor blade about the longitudinal axis defined by the length of the stem portion. In one particular aspect of the invention, a ratchet and pawl mechanism locks the stem in the rotatable retractor blade holder. Accordingly, the surgeon can easily set the distance to enable selective lateral adjustment of the retractor blade relative to the holder while still being able to rotate the stem about its longitudinal axis.

In another aspect of the present invention, the holder assembly includes a housing portion which houses at least a portion of the rotatable retractor blade holder. The housing allows the rotatable holder to be mounted onto another tilt mechanism which provides additional degrees of movement for the retractor blade when placed on the surgical frame. A releasable locking mechanism can be utilized to maintain the rotatable holder locked within the housing. The thumb actuator can be utilized in order to disengage the locking mechanism to allow the surgeon to easily rotate the stem portion and rotatable holder about the longitudinal axis to allow the retractor blade to be angularly adjusted in the operative site. The locking mechanism also can use biasing means which maintains the holder and stem portion in a locked position but which can be easily overcome by applying a rotational force to the stem portion to angularly adjust the retractor blade.

Other features and advantages of the present invention will become more apparent from the following detailed description of the invention when taken in conjunction with the accompanying exemplary drawings.

### BRIEF DESCRIPTION OF THE DRAWINGS

FIG. 1 is a perspective view of one particular embodiment of a rotatable retractor blade holder made in accordance with the present invention.

FIG. 2 is a side elevational view of the device depicted in FIG. 1.

FIG. 3 is an end view of the device depicted in FIG. 1 taken along lines 3-3.

FIG. 4 is a top view of the device depicted in FIG. 1.

FIG. 5 is a cross sectional view of the device depicted in FIG. 4 taken along lines 5-5.

FIG. 6 is a cross sectional view of the device depicted in FIG. 4 taken along lines 6-6.

FIG. 7 is a perspective view of a complete surgical retraction system which illustrates an embodiment of the present invention which is used to hold and position six surgical retractor blades on a surgical frame.

### DETAILED DESCRIPTION OF THE INVENTION

In one aspect, the present invention relates to locking mechanisms which can be used to hold a retractor blade that forms a portion of a surgical retractor assembly. For the sake of illustration, the following exemplary embodiments of the invention are directed to retractor blade holders used in typical surgical retraction systems, although it should be understood that the present invention can be applicable to other medical device applications as well.

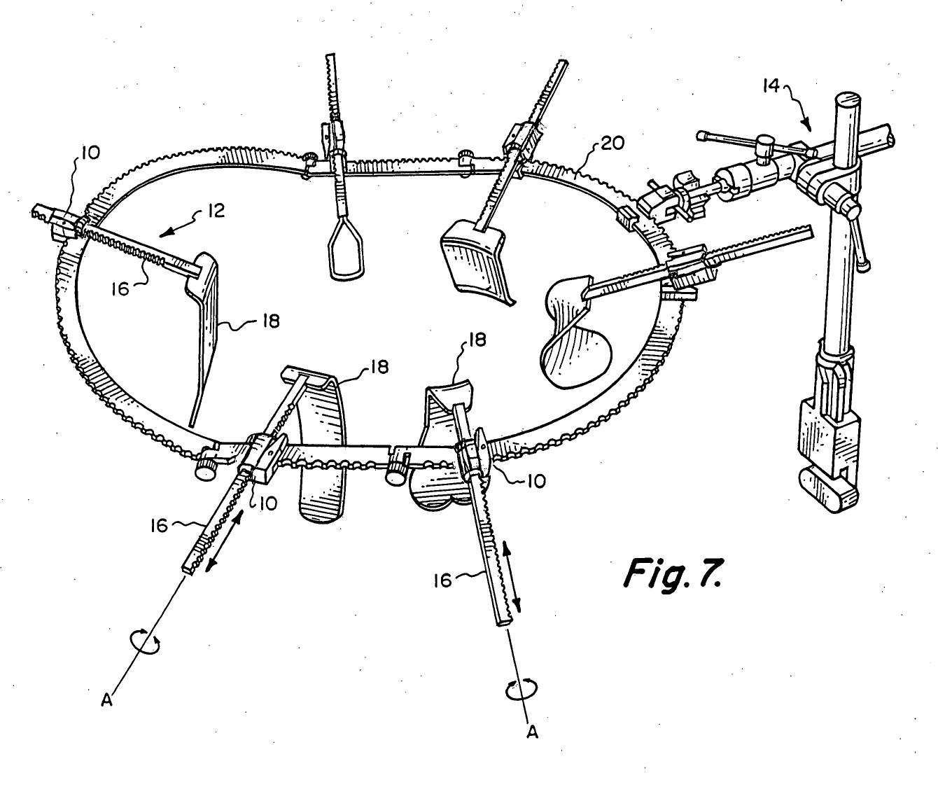

Referring specifically to FIGS. 1 and 7, one particular embodiment of a rotatable retractor blade holder 10 is shown. The retractor blade holder 10 provides multidimensional movement and placement of a composite retractor blade 12 on a typical surgical retractor assembly 14, shown in FIG. 7. The retractor blade 12 includes an elongate stem portion 16 and a surgical blade 18 used to engage and hold the patient's tissue. The retractor blade holder 10 with stem 16 and retractor blade 18 are mounted to a surgical frame 20 which forms part of the surgical retraction assembly 14. In use, the surgeon simply moves the stem 16 in a selected lateral movement (shown by arrows in FIGS. 1 and 7) to move the retractor blade 18 relative to the surgical site (not shown in FIG. 7). This lateral movement of the stem 16, in turn, adjusts the amount of tension placed on the outer margin of the patient's tissue in order to create an anatomical surgical opening in which the surgeon performs the particular surgery. The retractor blade holder 10 is also rotatable when mounted on the surgical frame 20 to allow the surgeon to rotate both the stem 16 and retractor blade 18 about the longitudinal axis (line A) of the stem 16 to position the retractor blade 18 as may be needed within the surgical site. Again, arrows in FIGS. 1 and 7 show the range of lateral movement and rotational movement which is possible when the retractor blade holder 10 of the present invention is mounted on the surgical frame 20.

Referring now specifically to FIG. 1, the retractor blade holder 10 of the present invention is shown attached to another tilt mechanism (shown in phantom) which supplies one or more additional degrees of freedom to allow the retractor blade holder 10 to be further tilted and/or rotated relative to the surgical site. Such a mechanism allows the surgeon to tilt both the stem 16 and the retractor blade 18 until the angle of inclination which is desired has been achieved. Additionally, such a mechanism could also provide additional rotation to the retractor blade holder in an axis different than the longitudinal axis (A) of the stem. Such a tilt mechanism is disclosed in U.S. Pat. No. 6,431,025, which is hereby incorporated by reference in its entirety. Such a mechanism also can include a component which enables the entire mechanism to be attached to the surgical frame 20 in a manner similar to that depicted in FIG. 7. It should be appreciated that this is just one of the type of tilt mechanism that can be utilized in accordance with the rotatable retractor blade holder 10 of the present invention.

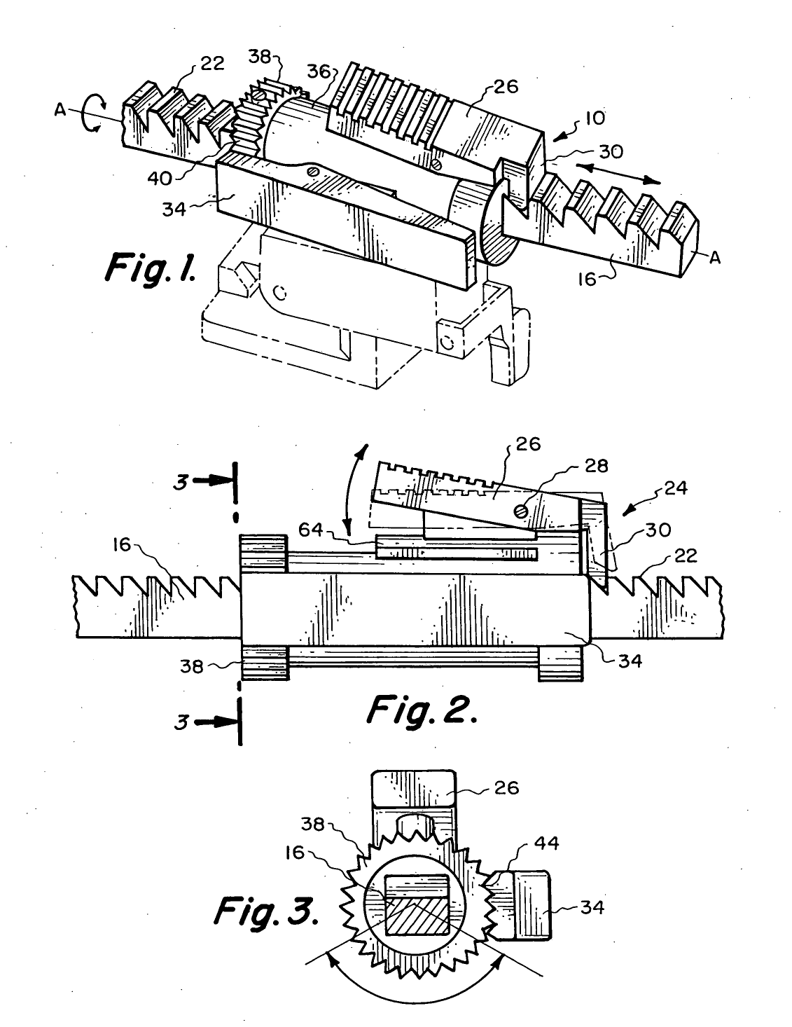

Referring now specifically to FIGS. 1-6, one particular embodiment of a retractor blade holder 10 made in accordance with the present invention is shown. The retractor blade holder 10 is designed to move the stem 16 of the composite retractor blade 12 in a lateral direction as is shown by the arrows in FIG. 1. As can be seen in FIG. 1, the surgical stem 16 has a series of teeth or ratchets 22 along one side which engages a pawl mechanism 24 that forms part of the retractor blade holder 10. This pawl mechanism 24 includes a thumb actuator 26 which is pivotally mounted onto the retractor blade holder by a pivot pin 28. A compression spring (not shown) biases the thumb actuator 26 to keep the thumb actuator 26 in a locked position until the thumb actuator 26 is pressed to release the pawl face 30 from engagement with the teeth 22 of the stem 16. FIG. 2 shows both the locked and unlocked position of this pawl mechanism 24. Arrows depicted in FIG. 2 show how the thumb actuator 26 can be moved in a downward fashion in order to disengage the pawl face 30 from the teeth 22 of the stem 16. This pawl mechanism 24 in conjunction with the teeth 22 of the stem 16 create a convenient mechanism which allows the surgeon to simply pull back on the stem portion as may be needed to apply the correct amount of tension to hold the retractor blade 18 within the margins of the surgical incision. As the stem 16 is pulled proximally away from the retractor blade holder 10, the pawl face 30 slides along the teeth 22 to engage another tooth 22 found on the stem 16. The pawl face 30 will remain engaged with the tooth 22 until the thumb actuator 26 is pushed down, which releases the pawl face 30 from engagement within the tooth 22, as is shown in phantom in FIG. 2. In this manner, if too much tension is placed on a particular retractor by the surgeon, the thumb actuator 26 can simply be quickly pressed to release the tension and allow the surgeon to pull back on the stem until the desired amount of tension can be placed on the composite retractor blade 12.

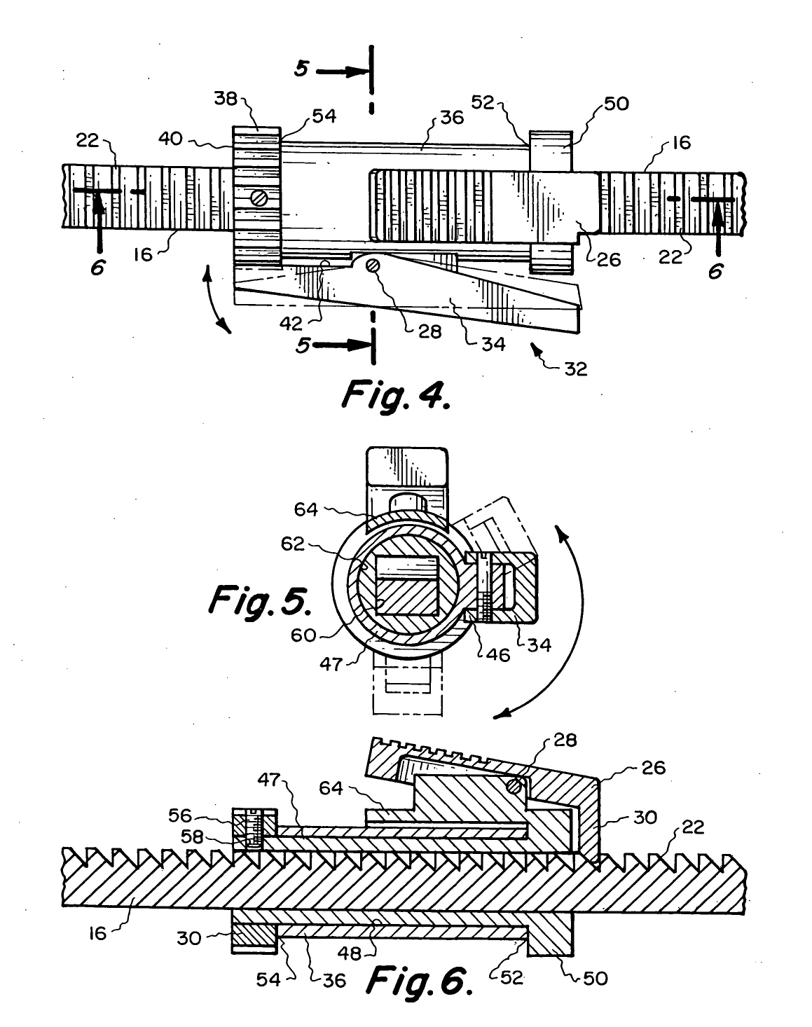

A second locking mechanism 32 is found on the retractor blade holder 10 which allows the rotation of the stem 16 and blade 18 along with the retractor blade holder 10. As can be seen in FIGS. 1-4, this second locking mechanism 32 includes a thumb actuator 34 mounted to a housing 36 in which the retractor blade holder 10 is rotatably mounted. This locking mechanism 32 further includes a ring 38 mounted to the retractor blade holder 10 that contains a number of indents 40 which extend circumferentially around the ring 38. This thumb actuator 34 has an engaging face 42 including at least one projection 44 designed to engage one or more of these indents 40 formed on the ring 38. FIG. 3 shows the engagement of a number of these projections 44 within a number of indents 40 formed on the ring 38. Like the other thumb actuator associated with the pawl mechanism 24, this thumb actuator 34 is pivotally mounted to the housing 36 by a pivot pin 28. Another compression spring (not shown) will bias this thumb actuator 34 in a locked position, keeping the projections 44 of the engaging face 42 in proper engagement with the indents 40 of the ring 38 until it is desired to rotate the retractor blade holder 10 along the longitudinal axis of the stem 16. In order to disengage the projections 44 from the indents 40, one merely needs to press down on the thumb actuator 34 which causes the engaging face 42 to move away from the ring 38, as is shown in phantom in FIG. 4. Additionally, the use of a biasing means with the thumb actuator 34 in conjunction with the locking mechanism 32 allows the stem 16 to be rotated about its longitudinal axis without the need to actuate the thumb actuator 34. In this regard, the compressing spring should be sufficiently strong to maintain a biasing force on the engaging face 42 against the ring 38 which can be overcome by simply applying a sufficient rotational force on the stem 16. In this regard, once a rotational force is applied to overcome the biasing force of the compression spring, the entire retractor blade holder 10 can be selectively rotated to a different position along the ring 38. The compression spring will then maintain the engaging face 42 in proper engaging contact with the ring 38 to maintain the desired angular position.

Referring now to FIG. 3, an end view of the rotator blade holder 10 which shows the ring 38 and a cross-section of the stem portion 16 is shown. Arrows show the degree of rotation that is usually needed in order to angularly adjust the retractor blade within the surgical site. Typically, the stem 16 and retractor blade holder 10 can be rotated from 0° to about 120° in order to obtain the necessary rotation needed in order to adjust the retractor blade 18 relative to the surgical site. The angular adjustment, of course, could be higher than from 0° to 120° degrees; however, if the stem is rotated too much, the retractor blade 18 will actually be rotated directly out the surgical site and out of engagement with the patient's tissue. Therefore, angular adjustment of about 0° to 120° is usually more than sufficient to achieve the necessary degree of rotation needed for the surgeon.

As is shown in FIG. 2, the housing 36 stores at least a portion of the retractor blade holder 10 and can be mounted onto a second tilt mechanism (shown in phantom in FIG. 1) and mounted to the surgical frame shown in FIG. 7. Alternatively, a simple mounting bracket could be attached to the housing to enable the rotatable retractor blade holder 10 to be mounted to the surgical frame. However, the use of an additional tilt mechanism, as referenced above, provides at least one additional degree of movement for the composite retractor blade 12 on the surgical frame.

As can be best seen in FIGS. 4-6, the housing 36 of this particular embodiment is shown as a cylindrical tubing having an internal lumen which receives at least a part of the retractor blade holder 10. The housing 36 includes a mounting bracket 46 upon which the thumb actuator 34 is pivotally mounted. The retractor blade holder 10 also can be made from a cylindrical tubing which forms a cylinder 47 that is slidingly disposed within the lumen 48 of the housing 36. Reference is made to FIG. 6 which shows the cross sectional relationship of the housing 36 with the retractor blade holder 10. At one end of the retractor blade holder 10, a second ring 50 extends outside of the lumen 48 of the housing 36, but contacts the end 52 of the housing 36. The opposite end of the retractor blade holder 10 is shown including the engaging ring 38 which, with the other ring 50, remains outside the lumen 48 of the housing 36 in an abutting relationship to the other end 54 of the housing 36. These two rings 38 and 50 prevent lateral movement of the retractor blade holder 10 within its housing 36 while still allowing the retractor blade holder 10 to rotate about the longitudinal axis of the stem 16. As is shown in FIG. 6, this ring 38 can be secured to the cylinder 47 which forms part of the retractor blade holder 10 utilizing a screw 56 which extends in an opening 58 in the cylinder 47. The other ring 50 can be similarly attached to the cylinder 47 utilizing screws or welding or other fastening techniques known in the art.

Referring now specifically to FIG. 5, the cylinder portion 47 of the retractor blade holder 10 includes a slot 60 formed in an insert 62 which is adapted to receive the stem 16. In this regard, the slot 60 of insert 62 is shown as having a relatively square cross-sectional opening which receives the stem portion 16. It should be appreciated that a second insert is located at the other end of the retractor blade holder 10 to help hold the stem 16 in position. In this fashion, a blade stem slot is created through the cylinder 47. These inserts 62 can be welded or otherwise attached to the cylinder 47 using techniques well known in the art. It also should be appreciated that although the stem is shown having a substantially square cross-section, different cross-sectional areas can be utilized in accordance with the present invention. Accordingly, the openings in the inserts forming part of the retractor blade holder 10 would be formed to receive the particular cross-sectional area of the stem.

Referring now to FIGS. 2, 5 and 6, it should be appreciated that a separate mounting structure 64 is needed in order to mount the thumb actuator 26 in place. Since the retractor blade holder 10 must rotate within the housing 36, this mounting structure 64 must rotate with the retractor blade holder 10 as well; otherwise, there would not be relative motion between the housing 36 and the retractor blade holder 10. Accordingly, this mounting structure 64 is shown attached to the ring 50 which forms part of the retractor blade holder 10 in this particular embodiment. As can best be seen in FIG. 6, the mounting structure 64 is detached from the housing 36 and can either be raised away from the surface of the housing 36 or it could be in sliding engagement with the outer surface of the housing 36. In this fashion, the blade retractor holder 10 remains slidably mounted within the lumen of the housing to permit the stem 16 and blade to be rotated about the longitudinal axis A. Other suitable ways of mounting the thumb actuator 26 also could be utilized without departing from the spirit and scope of the present invention.

The rotatable retractor blade holder can be made utilizing suitable biocompatible materials such as stainless steel or any another biocompatible material which is suitable for use. Preferably, the materials which are selected should be capable of being sterilized using known methods such as autoclaving.

While the invention has been illustrated and described herein, in terms of its use with clamping devices, it will be apparent to those skilled in the art that the device can take on a number of different forms and a number of different applications, both medical and non-medical. Other modifications and improvements may be made without departing from the scope of the invention.

What is claimed is:

1. A device for holding and positioning a retractor blade on a surgical frame, comprising:
a retractor blade holder adapted to receive a stem of a retractor blade, the retractor blade holder having a releasable locking mechanism to enable selective lateral adjustment of the retractor blade relative the retractor blade holder, the retractor blade holder being rotatable about the longitudinal axis of the stem when attached to the surgical frame.

2. The device of claim 1, wherein at least a portion of the retractor blade holder is disposed in a housing that allows the retractor blade holder to rotate about the longitudinal axis of the stem.

3. The device of claim 1, wherein the releasable locking mechanism is a pawl mechanism and the stem includes a plurality of teeth.

4. The device of claim 1, wherein the stem moves laterally in the retractor blade holder along the longitudinal axis of the stem.

5. The device of claim 2, further including an adjustable locking mechanism for maintaining the stem in a fixed position relative to the housing.

6. The device of claim 5, wherein the adjustable locking mechanism for maintaining the stem in a fixed position relative to the housing is a thumb actuator attached to the housing and an engaging component on the retractor blade holder.

7. The device of claim 6, wherein engaging component includes a plurality of indents extending circumferentially about the engaging component and the pivot thumb actuator includes at least one projection adapted to receive one of the indents to prevent rotational movement of the retractor blade holder.

8. The device of claim 1, wherein the retractor blade holder can rotate within a range of 90 degrees about the longitudinal axis of the stem.

9. The device of claim 2, wherein the releasable locking mechanism includes a thumb actuator attached to a mounting component on the retractor blade holder.

10. The device of claim 9, wherein the housing includes an opening adapted to receive a portion of the retractor blade holder and an outer surface, the mounting component being located outside of the housing.

11. The device of claim 9, wherein the mounting component moves rotationally with the retractor blade holder about the longitudinal axis of the stem.

12. A multi-position locking assembly for holding and positioning a retractor blade, comprising:
a retractor blade holder adapted to receive a stem of a retractor blade, the retractor blade holder having a releasable locking mechanism to enable selective lateral adjustment of the retractor blade relative the retractor blade holder along the longitudinal axis of the stem; and
a housing in which the retractor blade holder is rotatably mounted to allow the retractor blade holder to selectively rotate therein about the longitudinal axis of the stem.

13. The multi-position locking assembly of claim 12, wherein the releasable locking mechanism is a pawl mechanism.

14. The multi-position locking assembly of claim 13, wherein the stem includes a plurality of teeth which releasable lock with the pawl mechanism.

15. The multi-position locking assembly of claim 12, wherein housing includes a releasable locking mechanism attached thereto for releasably locking the retractor blade holder in a fixed position relative to the housing.

16. The multi-position locking assembly of claim 15, wherein the releasable locking mechanism for maintaining the stem in a fixed position relative to the housing is a thumb actuator attached to the housing and an engaging component on the retractor blade holder.

17. The multi-position locking assembly of claim 16, wherein engaging component includes a plurality of indents extending circumferentially about the engaging component and the pivot thumb actuator includes at least one projection adapted to receive one of the indents to prevent rotational movement of the retractor blade holder.

18. The multi-position locking assembly of claim 16, wherein engaging component is a ring having a plurality of indents extending circumferentially about the ring and the pivot thumb actuator includes at least one projection that receives an indent to lock the ring relative to the housing.

19. The multi-position locking assembly of claim 12, wherein the housing is adapted to be mounted to another device which allows the housing to be selectively moved in manner askew from the longitudinal axis of the stem.

20. The multi-position locking assembly of claim 12, wherein the releasable locking mechanism includes a thumb actuator attached to a mounting component on the retractor blade holder and the mounting component moves rotationally with the retractor blade holder about the longitudinal axis of the stem.
